(19) United States
(12) Patent Application Publication
(10) Pub. No.: US 2024/0116884 A1
(43) Pub. Date: Apr. 11, 2024

(54) Salts of Viloxazine

(71) Applicant: Apotex Inc., Toronto (CA)

(72) Inventors: Fabio E. S. Souza, Brantford (CA); Avedis Karadeolian, Brantford (CA); Allan W. Rey, Brantford (CA)

(21) Appl. No.: 18/371,093

(22) Filed: Sep. 21, 2023

Related U.S. Application Data
(60) Provisional application No. 63/409,255, filed on Sep. 23, 2022.

(51) Int. Cl. C07D 265/30

(52) U.S. Cl. CPC C07D 265/30; C07B 2200/13

(57) ABSTRACT

The present invention provides novel salts of viloxazine and crystalline forms thereof. Specific salts of viloxazine provided by the present invention include fumarate, hemi-DL-tartrate, naphthalene-2-sulfonate, and citrate. Also provided are pharmaceutical compositions including the viloxazine salts and crystalline forms thereof and the use of these salts in the treatment of attention-deficit hyperactivity disorder in a human subject suffering therefrom.

### CROSS-REFERENCE TO RELATED APPLICATION

This application claims priority to U.S. Provisional Patent Application No. 63/409,255 filed Sep. 23, 2022, the disclosure of which is hereby incorporated by reference in its entirety.

### BACKGROUND OF THE INVENTION

### Field of the Invention

The present invention is directed to novel salts of viloxazine and crystalline forms thereof, processes for the preparation thereof, pharmaceutical compositions containing these forms, and their use for the treatment of attention-deficit hyperactivity disorder (ADHD) in a human subject suffering therefrom.

### Description of Related Art

Viloxazine (1), or (±)-2-[(2-ethoxyphenoxy)methyl]morpholine is the active pharmaceutical ingredient (API) provided in the form of its hydrochloride salt (1:1) in branded pharmaceutical QELBREE®, a drug indicated for the treatment of attention-deficit hyperactivity disorder (ADHD) in adults and pediatric patients 6 years and older.

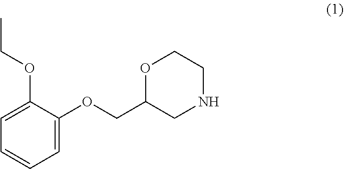 [SMILES: CCOc1ccccc1OCC1CNCCO1]

Viloxazine hydrogen oxalate salt is disclosed in U.S. Pat. No. 3,714,161 A, which discloses a family of compounds that are stated to be useful in the treatment of anxiety, neurotic states, and epilepsy. Further salts of viloxazine, including acetate and hydrochloride salts, are reported in, for example, U.S. Pat. Nos. 3,876,769 A, 3,712,890 A, WO 2011/130194 A1, and IN 202011003880 A. Salts of the (S) and (R) isomers of viloxazine with the respective (+) and (−) isomers of O,O-di-p-toluoyltartaric acid are reported in GB 1427097 A, while the (S) isomer of viloxazine with fumaric acid is prepared in U.S. Pat. No. 7,659,394 B2.

Different salt and/or crystalline forms of the same compound may have different crystal packing, thermodynamic, spectroscopic, kinetic, surface, and mechanical properties. For example, different salt and/or crystalline forms may have different stability properties such that a particular crystalline form may be more sensitive to temperature, relative humidity (RH), and/or light. Different salts and/or crystalline forms of a compound may also exhibit differences in physical characteristics such as flowability, density, and/or compressibility, properties relevant to handling and formulation. A particular salt and/or crystalline form may provide more favourable compressibility and/or density properties, thereby providing more desirable characteristics for formulation and/or product manufacturing. Particular salts and/or crystalline forms may also exhibit solubility differences with respect to one or more other substances present as impurities, providing for improved purification potential. The attributes of the salt and/or crystalline form of a drug substance used in the provision of high-drug load formulations in particular may factor more prominently into the performance of the drug product, given the relatively high proportion of drug substance present.

There exists a need for novel salts and crystalline forms of viloxazine having improved properties for use in providing drug products containing viloxazine, and commercially amenable processes for their manufacture.

### SUMMARY OF THE INVENTION

The present invention provides salts comprising viloxazine and an organic acid selected from the group consisting of fumaric acid, DL-tartaric acid, naphthalene-2-sulfonic acid, and citric acid. The organic acids used in the present invention are pharmaceutically acceptable acids.

The viloxazine salts and crystalline forms of the present invention exhibit form stability at high temperature and high humidity.

Accordingly, in a first aspect of the present invention, there is provided a fumarate salt of viloxazine. In a preferred embodiment of the first aspect, the molar ratio of viloxazine to fumaric acid is approximately 1:1. In a more preferred embodiment of the first aspect, the salt is characterized by a PXRD diffractogram comprising peaks, expressed in degrees 2θ (±0.2°), at 7.8°, 12.0°, and 24.1°. More preferably, the salt of the first aspect is characterized by a PXRD diffractogram further comprising at least three peaks, expressed in degrees 2θ (±0.2°), selected from the group consisting of: 6.9°, 11.5°, 15.7°, 16.5°, 21.0°, and 30.2°. In a further preferred embodiment of the first aspect, the PXRD diffractogram further comprises peaks, expressed in degrees 2θ (±0.2°), at 6.9°, 11.5°, 15.7°, 16.5°, 21.0°, and 30.2°. Preferably, the salt of the first aspect of the invention provides a PXRD diffractogram comprising peaks in substantially the same positions (±0.2° 2θ) as those shown in FIG. 1.

In a second aspect of the present invention, there is provided a DL-tartrate salt of viloxazine. In a preferred embodiment of the second aspect, the molar ratio of viloxazine to DL-tartaric acid is approximately 1:0.5. In a more preferred embodiment of the second aspect, the salt is characterized by a PXRD diffractogram comprising peaks, expressed in degrees 2θ (±0.2°), at 3.8°, 11.5°, and 12.8°. More preferably, the salt of the second aspect is characterized by a PXRD diffractogram further comprising at least three peaks, expressed in degrees 2θ (±0.2°), selected from the group consisting of: 6.9°, 9.5°, 13.8°, 16.3°, 19.7°, and 23.1°. In a further preferred embodiment of the second aspect, the PXRD diffractogram further comprises peaks, expressed in degrees 2θ (±0.2°), at 6.9°, 9.5°, 13.8°, 16.3°, 19.7°, and 23.1°. Preferably, the salt of the second aspect of the invention provides a PXRD diffractogram comprising peaks in substantially the same positions (±0.2° 2θ) as those shown in FIG. 2 and FIG. 3.

In a third aspect of the present invention, there is provided a naphthalene-2-sulfonate salt of viloxazine. In a preferred embodiment of the third aspect, the molar ratio of viloxazine to naphthalene-2-sulfonic acid is approximately 1:1. In a more preferred embodiment of the third aspect, the salt is characterized by a PXRD diffractogram comprising peaks, expressed in degrees 2θ (±0.2°), at 5.5°, 9.3°, and 16.5°. More preferably, the salt of the third aspect is characterized by a PXRD diffractogram further comprising at least three peaks, expressed in degrees 2θ (±0.2°), selected from the group consisting of: 7.2°, 10.9°, 11.9°, 15.6°, 18.6°, and 23.9°. In a further preferred embodiment of the third aspect, the PXRD diffractogram further comprises peaks, expressed in degrees 2θ (±0.2°), at 7.2°, 10.9°, 11.9°, 15.6°, 18.6°, and 23.9°. Preferably, the salt of the third aspect of the invention provides a PXRD diffractogram comprising peaks in substantially the same positions (±0.2° 2θ) as those shown in FIG. 4.

In a fourth aspect of the present invention, there is provided a citrate salt of viloxazine. In a preferred embodiment of the fourth aspect, the molar ratio of viloxazine to citric acid is approximately 1:1. In a more preferred embodiment of the fourth aspect, the salt is characterized by a PXRD diffractogram comprising peaks, expressed in degrees 2θ (±0.2°), at 6.0°, 10.3°, and 25.1°. More preferably, the salt of the fourth aspect is characterized by a PXRD diffractogram further comprising at least three peaks, expressed in degrees 2θ (±0.2°), selected from the group consisting of: 12.0°, 13.4°, 13.8°, 15.9°, 17.5°, and 19.7°. In a further preferred embodiment of the fourth aspect, the PXRD diffractogram further comprises peaks, expressed in degrees 2θ (±0.2°), at 12.0°, 13.4°, 13.8°, 15.9°, 17.5°, and 19.7°. Preferably, the salt of the fourth aspect of the invention provides a PXRD diffractogram comprising peaks in substantially the same positions (±0.2° 2θ) as those shown in FIG. 5.

In a fifth aspect of the present invention, there is provided a pharmaceutical composition comprising a salt of viloxazine according to the first, second, third, or fourth aspects of the invention, or a combination thereof, and one or more pharmaceutically acceptable excipients. Preferably, the pharmaceutical composition is in the form of a solid oral dosage form. Most preferably, the pharmaceutical composition is a capsule or a tablet. Preferably, the pharmaceutical composition of the fifth aspect comprises an amount of a viloxazine salt of the first, second, third, or fourth aspects that is equivalent to 100 mg of viloxazine free base.

In a sixth aspect of the present invention, there is provided the use of a salt of viloxazine according to the first, second, third, or fourth aspects of the invention, or a combination thereof, or the pharmaceutical composition of the fifth aspect of the invention, in the treatment of attention-deficit hyperactivity disorder.

In a seventh aspect of the present invention, there is provided a method of treating attention-deficit hyperactivity disorder comprising administering to a human subject in need thereof a therapeutically effective amount of a salt of viloxazine according to the first, second, third, or fourth aspects of the invention, or a combination thereof, or the pharmaceutical composition of the fifth aspect of the invention.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention are described, by way of example only, with reference to the attached Figures. The terms Fig., Figs., Figure, and Figures are used interchangeably in the specification to refer to the corresponding figures in the drawings.

FIG. 1 is a representative PXRD diffractogram of viloxazine fumarate Form APO-I as prepared in Example 1.

FIG. 2 is a representative PXRD diffractogram of viloxazine DL-tartrate Form APO-I as prepared in Example 2.

FIG. 3 is a truncated version of FIG. 2 having a condensed 2-theta range.

FIG. 4 is a representative PXRD diffractogram of viloxazine naphthalene-2-sulfonate Form APO-I as prepared in Example 3.

FIG. 5 is a representative PXRD diffractogram of viloxazine citrate Form APO-I as prepared in Example 5.

### DESCRIPTION OF THE INVENTION

The present invention provides novel salts of viloxazine and crystalline forms thereof providing improved properties over known salts of viloxazine.

The viloxazine salts and crystalline forms of the present invention exhibit differences in properties when compared to known salts of viloxazine. Depending on the specific salts and crystalline forms of the invention used, properties that differ between the invention and known salts of viloxazine include crystal packing properties such as molar volume, density, and hygroscopicity; thermodynamic properties such as melting point and solubility; kinetic properties such as dissolution rate and chemical/polymorphic stability; surface properties such as crystal habit/particle morphology; and/or mechanical properties such as hardness, tensile strength, compactibility, tabletting, handling, flow, and blending. The improved properties provided by the salts and crystalline forms of the present invention provide practical advantages over known forms of viloxazine that can be exploited to meet specific needs in the manufacture and formulation of viloxazine.

Depending on the manner in which the crystalline forms of the present invention are prepared, and the methodology and instrument used for PXRD analysis, the intensity of a given peak observed in a PXRD diffractogram of a crystalline form may vary when compared to the same peak in the representative PXRD diffractograms provided in

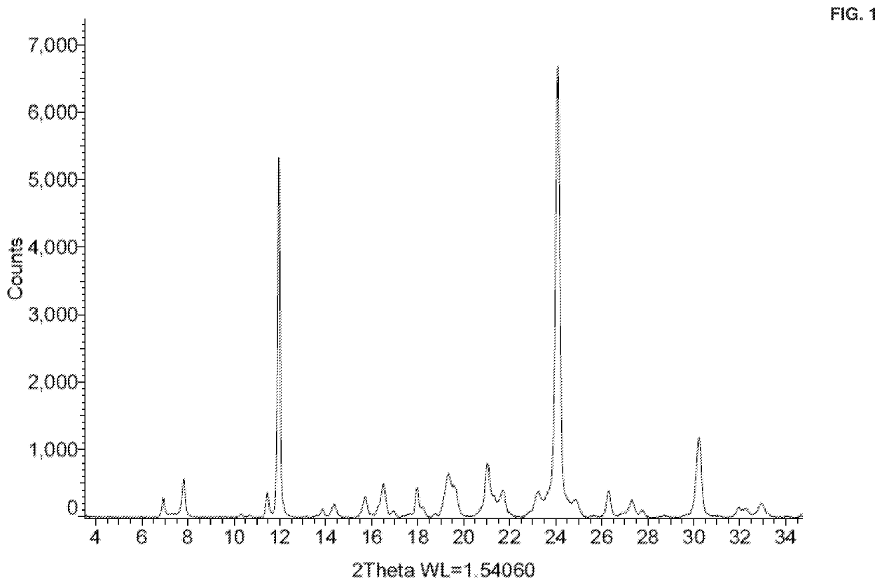

FIGS. 1 to 5. Thus, differences in relative peak intensities between peaks in a PXRD diffractogram for a given crystalline form may be observed when compared to the relative peak intensities of the peaks in the representative PXRD diffractograms of FIGS. 1 to 5. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the invention disclosed herein.

In addition to the differences in relative peak intensities that may be observed in comparison to the representative PXRD diffractograms provided in FIGS. 1 to 5, it is understood that individual peak positions may vary between ±0.2°2θ from the values observed in the representative PXRD diffractograms provided in FIGS. 1 to 5 for the crystalline forms of the invention, or listed in Tables 1 to 4. Such variations are known and understood by a person of skill in the art, and any such variations do not depart from the invention disclosed herein.

Further, depending on the instrument used for X-ray analysis and its calibration, uniform offsets in the peak position of each peak in a PXRD diffractogram of greater that 0.2° 2θ may be observed when compared to the representative PXRD diffractograms provided in FIGS. 1 to 5. Thus, PXRD diffractograms of the crystalline forms of the present invention may, in some circumstances, display the same relative peak positions as observed in the representative PXRD diffractograms provided in FIGS. 1 to 5, with the exception that each peak is offset in the same direction, and by approximately the same amount, such that the overall PXRD diffractogram is substantially the same in appearance as the PXRD diffractograms of FIGS. 1 to 5, with the exception of the uniform offset in peak positions. The observation of any such uniform peak shift in a PXRD diffractogram does not depart from the invention disclosed herein given that the relative peak positions of the individual peaks within the PXRD diffractogram remain consistent with the relative peak positions observed in the PXRD diffractograms of FIGS. 1 to 5.

As used herein, the term ‘crystalline form’ refers to a substance, particularly a

viloxazine salt, having a particular arrangement of its components in the crystal lattice, and which may be identified by physical characterization methods such as PXRD and/or DSC.

As used herein, the term “room temperature” refers to a temperature in the range of 20° C. to 25° C.

As used herein, the term “% w/w” (% weight/weight) refers to the ratio of the weight of a subject component to the weight of the subject mixture comprising that component, expressed as a percentage. For example, with respect to a pharmaceutical composition comprising a mixture of components, % w/w refers to the ratio of the weight of a component to the weight of the composition, expressed as a percentage.

As used herein, an “extended-release” dosage form refers to a dosage form which, when taken orally, substantially provides at least 80% of the drug in a form available to be absorbed in a time of at least 2 hours. For example, the pharmaceutical compositions of the present invention are preferably provided as extended-release solid oral capsule dosage forms. However, the compositions may be provided in other release dosage forms.

When describing the embodiments of the present invention there may be a common variance to a given temperature or time that would be understood or expected by the person skilled in the art to provide substantially the same result. For example, when reference is made to a particular temperature, it is to be understood by the person skilled in the art that there is an allowable variance of ±5° C. associated with that temperature. When reference is made to a particular time, it is to be understood that there is an allowable variance of ±10 minutes when the time is one or two hours, and ±1 hour when longer periods of time are referenced.

In a first embodiment of the present invention, there is provided a new salt of viloxazine, viloxazine fumarate Form APO-I, wherein the molar ratio of viloxazine to fumaric acid is approximately 1:1.

Viloxazine fumarate Form APO-I can be characterized by a PXRD diffractogram comprising, among other peaks, characteristic peaks, expressed in degrees 2θ (±0.2°), at 7.8°, 12.0°, and 24.1°. Preferably, the PXRD diffractogram further comprises at least three peaks, expressed in degrees 2θ (±0.2°), selected from the group consisting of 6.9°, 11.5°, 15.7°, 16.5°, 21.0°, and 30.2°. More preferably, the PXRD diffractogram further comprises peaks, expressed in degrees 2θ (±0.2°), at 6.9°, 11.5°, 15.7°, 16.5°, 21.0°, and 30.2°. PXRD studies of uncapped samples of viloxazine fumarate Form APO-I maintained in a 40° C./75% RH stability chamber for at least 3 weeks showed that no change in the crystalline form occurred.

An illustrative PXRD diffractogram of viloxazine fumarate Form APO-I, as prepared in Example 1, is shown in FIG. 1. A peak listing, comprising representative peaks from the PXRD diffractogram in FIG. 1, and their relative intensities, is provided in Table 1. Although illustrative of the PXRD diffractogram that is provided for the viloxazine fumarate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 1
Relative peak intensities of viloxazine
fumarate Form APO-I from FIG. 1

| Angle (2θ) | Relative intensity (%) |
|---|---|
| 6.93 | 4.1 |
| 7.83 | 8.1 |
| 11.47 | 5.5 |
| 11.96 | 77.6 |
| 14.38 | 3.0 |
| 15.71 | 4.3 |
| 16.50 | 7.3 |
| 17.99 | 6.2 |
| 19.35 | 9.4 |
| 21.03 | 11.7 |
| 24.09 | 100.0 |
| 26.30 | 5.9 |
| 30.24 | 17.1 |

In a second embodiment of the present invention, there is provided a new salt of viloxazine, viloxazine hemi-DL-tartrate Form APO-I, wherein the molar ratio of viloxazine to DL-tartaric acid is approximately 1:0.5.

Viloxazine hemi-DL-tartrate Form APO-I can be characterized by a PXRD diffractogram comprising, among other peaks, characteristic peaks, expressed in degrees 2θ (±0.2°), at 3.8°, 11.5°, and 12.8°. Preferably, the PXRD diffractogram further comprises at least three peaks, expressed in degrees 2θ (±0.2°), selected from the group consisting of 6.9°, 9.5°, 13.8°, 16.3°, 19.7°, and 23.1°. More preferably, the PXRD diffractogram further comprises peaks, expressed in degrees 2θ (±0.2°), at 6.9°, 9.5°, 13.8°, 16.3°, 19.7°, and 23.1°. PXRD studies of uncapped samples of viloxazine hemi-DL-tartrate Form APO-I maintained in a 40° C./75% RH stability chamber for at least 3 weeks showed that no change in the crystalline form occurred.

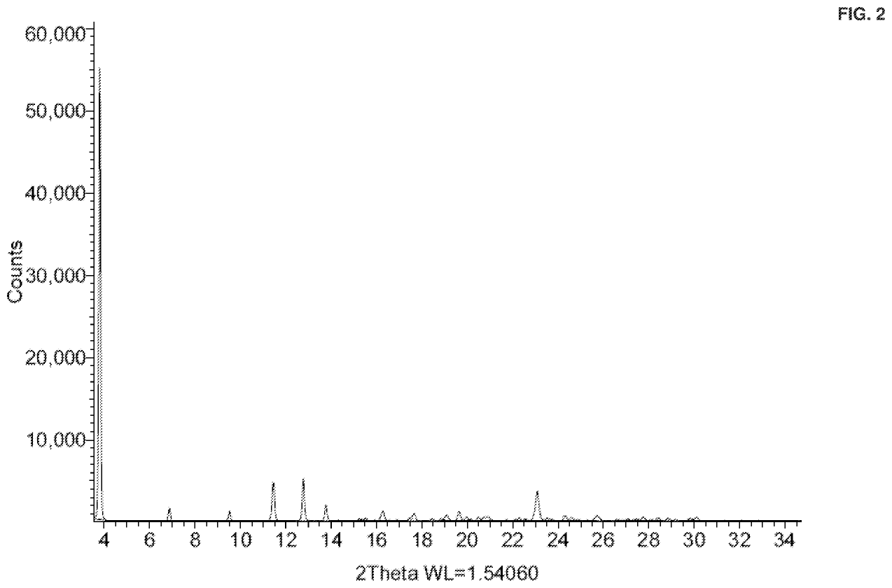

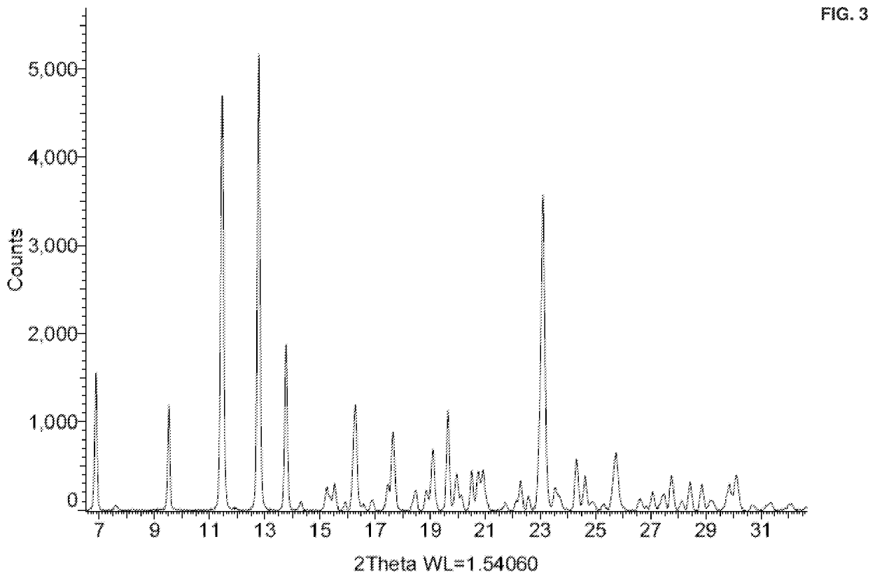

An illustrative PXRD diffractogram of viloxazine hemi-DL-tartrate Form APO-I, as prepared in Example 2, is shown in FIG. 2. A truncated version of the PXRD diffractogram shown in FIG. 2 is provided in FIG. 3, wherein the scale is adjusted to enlarge the intensity of the higher angle peaks. A peak listing, comprising representative peaks from the PXRD diffractogram in FIG. 2, and their relative intensities, is provided in Table 2. Although illustrative of the PXRD diffractogram that is provided for the viloxazine hemi-DL-tartrate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 2
Relative peak intensities of viloxazine
hemi-DL-tartrate Form APO-I from FIG. 2

| Angle (2θ) | Relative intensity (%) |
|---|---|
| 3.81 | 100.0 |
| 6.88 | 2.9 |
| 9.52 | 2.3 |
| 11.46 | 8.8 |
| 12.78 | 9.9 |
| 13.78 | 3.6 |
| 16.29 | 2.3 |
| 17.66 | 1.7 |
| 19.10 | 1.3 |
| 19.65 | 2.3 |
| 20.51 | 0.9 |
| 23.10 | 7.0 |
| 25.74 | 1.3 |

In a third embodiment of the present invention, there is provided a new salt of viloxazine, viloxazine naphthalene-2-sulfonate Form APO-I, wherein the molar ratio of viloxazine to naphthalene-2-sulfonic acid is approximately 1:1.

Viloxazine naphthalene-2-sulfonate Form APO-I can be characterized by a PXRD diffractogram comprising, among other peaks, characteristic peaks, expressed in degrees 2θ (±0.2°), at 5.5°, 9.3°, and 16.5°. Preferably, the PXRD diffractogram further comprises at least three peaks, expressed in degrees 2θ (±0.2°), selected from the group consisting of 7.2°, 10.9°, 11.9°, 15.6°, 18.6°, and 23.9°. More preferably, the PXRD diffractogram further comprises peaks, expressed in degrees 2θ (±0.2°), at 7.2°, 10.9°, 11.9°, 15.6°, 18.6°, and 23.9°. PXRD studies of uncapped samples of viloxazine naphthalene-2-sulfonate Form APO-I maintained in a 40° C./75% RH stability chamber for at least 3 weeks showed that no change in the crystalline form occurred.

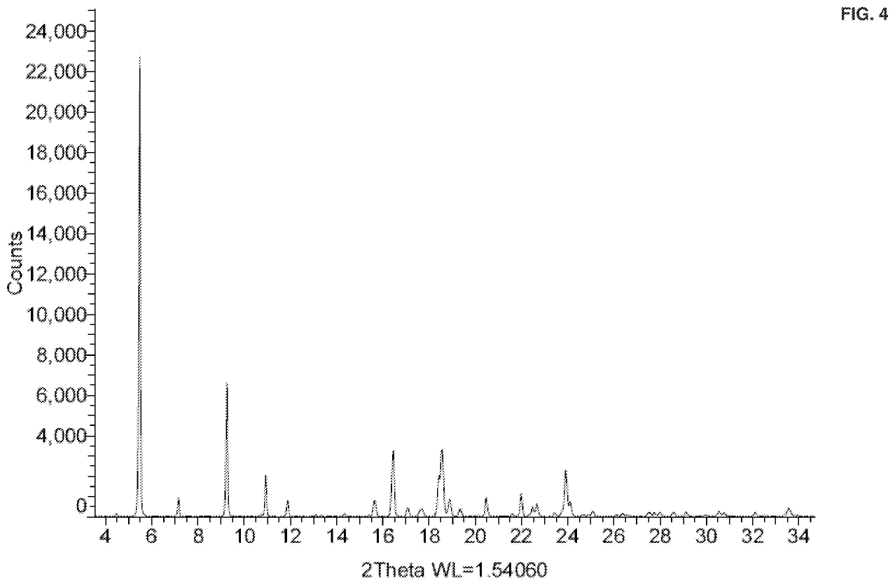

An illustrative PXRD diffractogram of viloxazine naphthalene-2-sulfonate Form APO-I, as prepared in Example 3, is shown in FIG. 4. A peak listing, comprising representative peaks from the PXRD diffractogram in FIG. 4, and their relative intensities, is provided in Table 3. Although illustrative of the PXRD diffractogram that is provided for the viloxazine naphthalene-2-sulfonate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 3
Relative peak intensities of viloxazine naphthalene-
2-sulfonate Form APO-I from FIG. 4

| Angle (2θ) | Relative intensity (%) |
|---|---|
| 5.47 | 100.0 |
| 7.16 | 4.1 |
| 9.25 | 30.1 |
| 10.94 | 9.4 |
| 11.88 | 3.7 |
| 15.64 | 3.6 |
| 16.45 | 15.0 |
| 18.56 | 14.4 |
| 20.47 | 5.0 |
| 21.98 | 6.0 |
| 23.91 | 11.5 |

In a fourth embodiment of the present invention, there is provided a new salt of viloxazine, viloxazine citrate Form APO-I, wherein the molar ratio of viloxazine to citric acid is approximately 1:1.

Viloxazine citrate Form APO-I can be characterized by a PXRD diffractogram comprising, among other peaks, characteristic peaks, expressed in degrees 2θ (±0.2°), at 6.0°, 10.3°, and 25.1°. Preferably, the PXRD diffractogram further comprises at least three peaks, expressed in degrees 2θ (±)0.2°, selected from the group consisting of 12.0°, 13.4°, 13.8°, 15.9°, 17.5°, and 19.7°. More preferably, the PXRD diffractogram further comprises peaks, expressed in degrees 2θ (±0.2°), at 12.0°, 13.4°, 13.8°, 15.9°, 17.5°, and 19.7°.

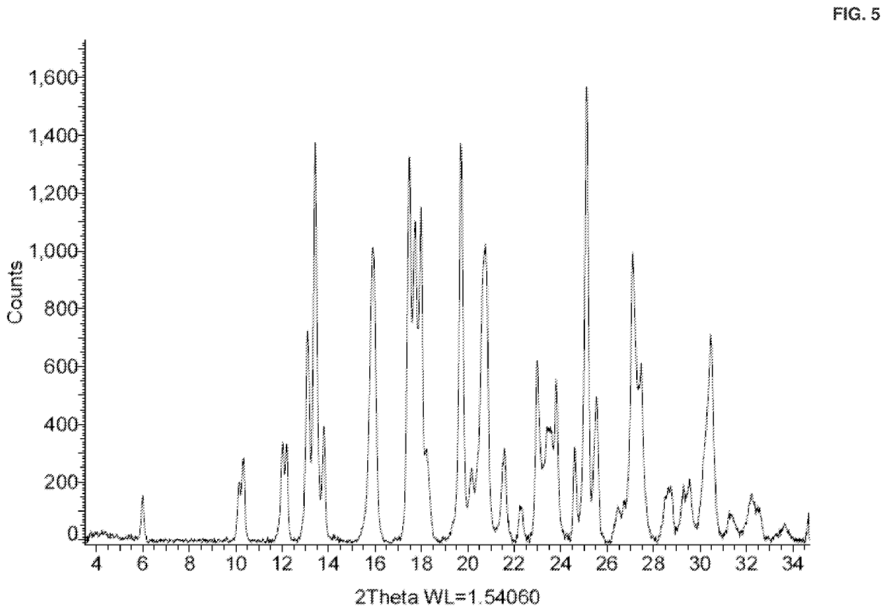

An illustrative PXRD diffractogram of viloxazine citrate Form APO-1, as prepared in Example 5 is shown in FIG. 5. A peak listing, comprising representative peaks from the PXRD diffractogram in FIG. 5, and their relative intensities, is provided in Table 4. Although illustrative of the PXRD diffractogram that is provided for the viloxazine citrate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 4
Relative peak intensities of viloxazine
citrate Form APO-I from FIG. 5

| Angle (2θ) | Relative intensity (%) |
|---|---|
| 6.00 | 8.8 |
| 10.15 | 11.5 |
| 10.34 | 16.0 |
| 12.03 | 20.0 |
| 12.20 | 19.5 |
| 13.09 | 41.5 |
| 13.42 | 80.5 |
| 13.80 | 23.3 |
| 15.91 | 59.9 |
| 17.49 | 78.0 |
| 17.73 | 63.0 |
| 17.98 | 68.6 |
| 19.70 | 83.6 |
| 20.76 | 59.2 |
| 21.59 | 19.2 |
| 22.98 | 38.9 |
| 23.81 | 35.0 |
| 24.61 | 20.2 |
| 25.12 | 100.0 |
| 25.54 | 30.1 |
| 27.11 | 60.7 |
| 30.46 | 44.7 |

In a fifth embodiment of the invention, there is provided a pharmaceutical composition comprising one or more viloxazine salt(s) selected from the group consisting of viloxazine fumarate, viloxazine hemi-DL-tartrate, viloxazine naphthalene-2-sulfonate, viloxazine citrate, and combinations thereof, with one or more pharmaceutically acceptable excipients. Preferably, the pharmaceutical composition comprises one or more crystalline form(s) of a viloxazine salt selected from the group consisting of viloxazine fumarate Form APO-I, viloxazine hemi-DL-tartrate Form APO-I, viloxazine naphthalene-2-sulfonate Form APO-I, viloxazine citrate Form APO-I, and combinations thereof, with one or more pharmaceutically acceptable excipients. The amount of viloxazine salt(s) or crystalline form(s) present in the composition, expressed as the equivalent viloxazine free base, is preferably from about 25% w/w to about 75% w/w, preferably from about 25% w/w to about 50% w/w, the remainder comprising pharmaceutically acceptable excipients. Preferably, the pharmaceutical composition is a solid dosage form suitable for oral administration, such as a capsule, tablet, pill, powder, or granulate. More preferably, the pharmaceutical composition is a capsule, most preferably an extended-release capsule. Preferably, the pharmaceutical composition provides a dose of one or more viloxazine salt(s) selected from the group consisting of viloxazine fumarate, viloxazine hemi-DL-tartrate, viloxazine naphthalene-2-sulfonate, and viloxazine citrate such that the total is equivalent to from about 80 mg to about 800 mg viloxazine free base, particularly the composition provides a dose that is equivalent to the 100 mg, 150 mg, or 200 mg of viloxazine free base found in QELBREE® drug products.

Suitable pharmaceutically acceptable excipients are preferably inert with respect to the viloxazine salts of the present invention and comprise from about 25% w/w to about 75% w/w of the composition. Suitable pharmaceutically acceptable excipients may include, for example, one or more excipients selected from binders such as lactose, starches, modified starches, sugars (e.g. sucrose), gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copovidone, gelatine, polyvinylpyrrolidone (PVP), and sodium alginate; fillers or diluents such as lactose, sugars (e.g. sucrose), starches, modified starches, mannitol, sorbitol, inorganic salts, cellulose derivatives (e.g., microcrystalline cellulose, cellulose), calcium sulphate, xylitol, and lactitol; disintegrants such as croscarmellose sodium, crospovidone, polyvinylpyrrolidone, sodium starch glycollate, corn starch, microcrystalline cellulose, hydroxypropyl methylcellulose, and hydroxypropyl cellulose; lubricants such as magnesium stearate, magnesium lauryl stearate, sodium stearyl fumarate, stearic acid, calcium stearate, zinc stearate, potassium benzoate, sodium benzoate, myristic acid, palmitic acid, mineral oil, hydrogenated castor oil, medium-chain triglycerides, Poloxamer, polyethylene glycol, and talc; and dispersants or solubility enhancing agents, such cyclodextrins, glyceryl monostearate, hypromellose, meglumine, Poloxamer, polyoxyethylene castor oil derivatives, polyoxyethylene stearates, polyoxylglycerides, povidone, and stearic acid. Other excipients including preservatives, stabilisers, anti-oxidants, silica flow conditioners, antiadherents, or glidants may be added as required. Other suitable excipients and the preparation of solid oral dosage forms are well known to person of skill in the art, and is described generally, for example, in Remington The Science and Practice of Pharmacy 21^{st} Edition (Lippincott Williams & Wilkins: Philadelphia; 2006; Chapter 45).

Optionally, when the pharmaceutical compositions are solid dosage forms, the solid dosage forms may be prepared with coatings, such as enteric coatings and extended-release coatings. Exemplary excipients for use in extended-release coatings include waxes, glyceryl monostearate, stearic acid, palmitic acid, glyceryl monopalmitate, ethylcellulose, cellulose acetate, hydroxypropyl cellulose, hydroxypropyl methyl cellulose, methyl cellulose, acrylic resins, povidone, carrageenan, carboxymethylcellulose, polyvinyl alcohol, xanthan gum, and combinations thereof. Exemplary excipients for use in enteric coatings include cellulose acetate phthalate, polyvinyl acetate phthalate, hydroxypropyl methylcellulose phthalate, acrylic copolymers, carboxymethyl ethylcellulose, hydroxypropyl methylcellulose acetate succinate, and combinations thereof. Preferably, the enteric and/or extended-release coatings comprise from about 5% w/w to about 65% w/w of the composition. Such coatings, and their application, are well known to persons skilled in the art, and are described, for example, in Remington The Science and Practice of Pharmacy 21^{st} Edition (Lippincott Williams & Wilkins: Philadelphia; 2006; Chapter 46).

In a sixth embodiment of the invention, there is provided a method of treating attention-deficit hyperactivity disorder (ADHD) comprising administering to a human subject in need thereof one or more viloxazine salt(s) selected from the group consisting of viloxazine fumarate, viloxazine hemi-DL-tartrate, viloxazine naphthalene-2-sulfonate, viloxazine citrate and combinations thereof, and a pharmaceutically acceptable excipient.

Preferably, the method of treatment comprises administering one or more crystalline form(s) of a viloxazine salt selected from the group consisting of viloxazine fumarate Form APO-I, viloxazine hemi-DL-tartrate Form APO-I, viloxazine naphthalene-2-sulfonate Form APO-I, viloxazine citrate Form APO-I, and combinations thereof, and a pharmaceutically acceptable excipient. Methods of treatment comprising administering a therapeutically effective amount of viloxazine in the treatment of ADHD have been disclosed in, for example, WO 2010/028207 A2.

### EXAMPLES

The following non-limiting examples are illustrative of some of the aspects and embodiments of the invention described herein.

The viloxazine free base used as a starting material in the following examples was commercially available and in the form of an oil.

### PXRD Analysis

PXRD diffractograms were recorded on a Bruker D8 Discover powder X-ray diffractometer (Bruker AXS LLC, Karlsruhe, Germany). The generator was a Incoatec Microfocus Source (IμS) Cu tube (λ=1.54060 Å) with a voltage of 50 kV and current of 1.00 mA, using a divergence slit of 0.1 mm and collimator of 2.0 mm. For each sample, two frames were collected using a still scan with a PILATUS3 R 100K-A detector at the distance of 294.2 mm from the sample. Raw data were evaluated using the program DIFFRAC.EVA (Bruker AXS LLC, Karlsruhe, Germany).

### Example 1: Preparation of Viloxazine Fumarate Form APO-I

To a suspension of fumaric acid (196 mg) in methanol (0.9 mL) and water (0.1 mL) was added a solution of viloxazine free base (400 mg) in acetone (5 mL). The resulting solution was heated to 50° C. for 30 minutes, allowed to cool to room temperature, and stirred for approximately 16 hours. The precipitated solids were collected by filtration, washed with acetone (2×0.9 mL), and dried in vacuo at room temperature for 24 hours to afford viloxazine fumarate Form APO-I (294 mg) as a white solid. The PXRD of the sample is shown in FIG. 1.

¹H NMR (500 MHz, DMSO-d₆) δ6.94-6.98 (m, 2H), 6.84 - 6.92 (m, 2H), 6.51 (s, 2H), 4.01 (q, J=7.0 Hz, 2H), 3.98 (dd, J=5.5, 10.6 Hz, 1H), 3.93 (dd, J=4.4, 10.5 Hz, 1H), 3.86-3.92 (m, 2H), 3.63 (td, J=2.6, 11.9 Hz, 1H), 3.14 (br d, J=11.8 Hz, 1H), 2.97 (br d, J=12.6 Hz, 1H), 2.83 (td, J=3.6, 12.2 Hz, 1H), 2.76 (dd, J=10.6, 12.5 Hz, 1H), 1.32 (t, J=7.0 Hz, 3H).

### Example 2: Preparation of Viloxazine Hemi-DL-Tartrate Form APO-1

To a suspension of DL-tartaric acid (253 mg) in methanol (0.9 mL) and water (0.1 mL) was added a solution of viloxazine free base (400 mg) in acetone (5 mL). The resulting solution was heated to 50° C. for 30 minutes, allowed to cool to room temperature, and stirred for approximately 16 hours. The precipitated solids were collected by filtration, washed with acetone (2×0.9 mL), and dried in vacuo at room temperature for 24 hours to afford viloxazine hemi-DL-tartrate Form APO-I (265 mg) as a white solid. PXRD of the sample is shown in FIGS. 2 and 3.

¹H NMR (500 MHz, DMSO-d₆) δ6.95-6.98 (m, 2H), 6.84-6.91 (m, 2H), 4.01 (q, J=7.0 Hz, 2H), 3.97 (dd, J=5.7, 10.5 Hz, 1H), 3.92 (dd, J=4.5, 10.5 Hz, 1H), 3.91 (s, 1H), 3.82-3.88 (m, 2H), 3.60 (td, J=2.6, 11.7 Hz, 1H), 3.10 (br d, J=11.5 Hz, 1H), 2.93 (br d, J=12.5 Hz, 1H), 2.80 (td, J=3.5, 12.1 Hz, 1H), 2.72 (dd, J=10.6, 12.4 Hz, 1H), 1.32 (t, J=6.9 Hz, 3H).

### Example 3: Preparation of Viloxazine Naphthalene-2-Sulfonate Form APO-1

To a solution of naphthalene-2-sulfonic acid hydrate (382 mg) in methanol (0.9 mL) and water (0.1 mL) was added a solution of viloxazine free base (400 mg) in acetone (5 mL). The resulting solution was heated to 50° C. for 30 minutes, allowed to cool to room temperature, and stirred for approximately 16 hours. Very little precipitation occurred, and methyl t-butyl ether (6 mL) was added. After stirring for 3 hours at room temperature, the solids were collected by filtration, washed with acetone (2×0.9 mL), and dried in vacuo at room temperature for 24 hours to afford viloxazine naphthalene-2-sulfonate Form APO-I (526 mg) as a white solid. The PXRD of the sample is shown in FIG. 4.

¹H NMR (500 MHz, DMSO-d₆) δ8.90 (s, 2H), 8.17 (d, J=1.6 Hz, 1H), 7.96-8.00 (m, 1H), 7.89-7.93 (m, 1H), 7.88 (d, J=8.6 Hz, 1H), 7.73 (dd, J=8.5, 1.7 Hz, 1H), 7.51-7.55 (m, 2H), 6.96-6.99 (m, 2H), 6.85-6.94 (m, 2H), 4.01 (q, J=7.0 Hz, overlapping multiplet, 6H), 3.74 (td, J=12.4, 2.4 Hz, 1H), 3.38 (br d, J=12.8 Hz, 1H), 3.25 (br d, J=12.9 Hz, 1H), 2.97-3.07 (m, 2H), 1.32 (t, J=7.0 Hz, 3H).

### Example 4: Preparation of Viloxazine Citrate Form APO-I Seeds

Citric acid (157.1 mg) and viloxazine (227.3 mg) were each dissolved in 1 mL of methanol, and the solutions were combined. One drop of water was added to the reaction mixture, and the solvent was allowed to evaporate over 96 hours. To the resulting oil was added dichloromethane (3.5 mL) along with two drops of water, and the sample was sonicated three times for 15 minutes each, after which the solvent was removed by a rotary evaporator and the resulting oil was placed under vacuum (˜1 Torr) for 72 hours. Another portion (3.5 mL) of dichloromethane was added to the sample, and after 15 minutes of sonication, the supernatant liquid was decanted. The remaining sticky/waxy sample slowly solidified over time while sitting under ambient conditions (with perturbation of the sample at the 14- and 15-day mark). After one month sitting under ambient conditions, viloxazine citrate Form APO-I (157.6 mg) was obtained as a crystalline white powder.

### Example 5: Preparation of Viloxazine Citrate Form APO-1

To a stirred solution of citric acid (95 mg) in acetone (4 mL) was added viloxazine free base (113 mg) in one portion. The mixture became immediately cloudy, followed by separation of an oily material, which was seeded with a small amount (5 mg) of the viloxazine citrate Form APO-I prepared in Example 4. The resulting mixture was stirred vigorously for approximately 28 hours, during which the oil solidified into a free-flowing powder. The solids were collected by filtration, washed once with acetone (1 mL), and dried in vacuo at room temperature to afford viloxazine citrate Form APO-I as a crystalline white powder (199 mg). The PXRD of the sample is shown in FIG. 5.

¹H NMR (500 MHz, DMSO-d₆) δ9.69 (v br s), 6.94-7.02 (m, 2H), 6.82-6.94 (m, 2H), 3.92-4.04 (m, 6H), 3.68 (td, J=2.3, 12.1 Hz, 1H), 3.26 (br d, J=12.3 Hz, 1H), 3.10 (br d, J=12.7 Hz, 1H), 2.93 (td, J=3.7, 12.4 Hz, 1H), 2.88 (dd, J=10.6, 12.5 Hz, 1H), 2.56 (d, J=15.1 Hz, 2H), 2.49 (d, J=15.0 Hz, 2H), 1.33 (t, J=7.0 Hz, 3H).

What is claimed is:

1. A fumarate salt of viloxazine.

2. The fumarate salt of viloxazine of claim 1, wherein the molar ratio of viloxazine to fumaric acid is approximately 1:1.

3. The fumarate salt of claim 2, characterized by a PXRD diffractogram comprising peaks, expressed in degrees 2θ (±0.2°), at 7.8°, 12.0°, and 24.1°.

4. The fumarate salt of claim 3, further comprising at least three peaks in the PXRD diffractogram, expressed in degrees 2θ (±0.2°), selected from the group consisting of: 6.9°, 11.5°, 15.7°, 16.5°, 21.0°, and 30.2°.

5. The fumarate salt of claim 3, further comprising peaks in the PXRD diffractogram, expressed in degrees 2θ (±0.2°), at 6.9°, 11.5°, 15.7°, 16.5°, 21.0°, and 30.2°.

6. The fumarate salt of claim 1, providing a PXRD diffractogram comprising peaks in substantially the same positions (±0.2° 2θ) as those shown in FIG. 1.

7. A salt of viloxazine selected from the group consisting of:
a) a DL-tartrate salt of viloxazine;
b) a naphthalene-2-sulfonate salt of viloxazine; and
c) a citrate salt of viloxazine.

8. The DL-tartrate salt of viloxazine of claim 7, wherein the molar ratio of viloxazine to DL-tartaric acid is approximately 1:0.5.

9. The DL-tartrate salt of claim 8, characterized by a PXRD diffractogram comprising peaks, expressed in degrees 2θ (±0.2°), at 3.8°, 11.5°, and 12.8°.

10. The DL-tartrate salt of claim 9, further comprising at least three peaks in the PXRD diffractogram, expressed in degrees 2θ (±0.2°), selected from the group consisting of: 6.9°, 9.5°, 13.8°, 16.3°, 19.7°, and 23.1°.

11. The naphthalene-2-sulfonate salt of viloxazine of claim 7, wherein the molar ratio of viloxazine to naphthalene-2-sulfonic acid is approximately 1:1.

12. The naphthalene-2-sulfonate salt of claim 11, characterized by a PXRD diffractogram comprising peaks, expressed in degrees 2θ (±0.2°), at 5.5°, 9.3°, and 16.5°.

13. The naphthalene-2-sulfonate salt of claim 12, further comprising at least three peaks in the PXRD diffractogram, expressed in degrees 2θ (±0.2°), selected from the group consisting of: 7.2°, 10.9°, 11.9°, 15.6°, 18.6°, and 23.9°.

14. The citrate salt of viloxazine of claim 7, wherein the molar ratio of viloxazine to citric acid is approximately 1:1.

15. The citrate salt of claim 14, characterized by a PXRD diffractogram comprising peaks, expressed in degrees 2θ (±0.2°), at 6.0°, 10.3°, and 25.1°.

16. The citrate salt of claim 14, further comprising at least three peaks in the PXRD diffractogram, expressed in degrees 2θ (±0.2°), selected from the group consisting of: 12.0°, 13.4°, 13.8°, 15.9°, 17.5°, and 19.7°.

17. A pharmaceutical composition comprising the salt of viloxazine according to claim 1, and one or more pharmaceutically acceptable excipients.

18. The pharmaceutical composition of claim 17, wherein the pharmaceutical composition is a capsule.

19. A method of treating attention-deficit hyperactivity disorder comprising administering the pharmaceutical composition of claim 18 to a human subject in a therapeutically effective amount for the treatment of attention-deficit hyperactivity disorder.
